(19) United States
(12) Patent Application Publication
(10) Pub. No.: US 2026/0098010 A1
(43) Pub. Date: Apr. 9, 2026

(54) ß-CAROTENE SYNTHESIS (I)

(71) Applicant: DSM IP Assets B.V., Maastricht (NL)

(72) Inventors: Leonardo LE, Kaiseraugst (CH); Christian LOTHSCHUETZ, Kaiseraugst (CH)

(21) Appl. No.: 19/114,963

(22) PCT Filed: Sep. 25, 2023
(86) PCT No.: PCT/EP2023/076342

§ 371 Date: Mar. 25, 2025

(30) Foreign Application Priority Data
Sep. 29, 2022 (EP) 22020467.1

(51) Int. Cl. C07C 403/24

(52) U.S. Cl. CPC C07C 403/24; C07C 2601/16

(57) ABSTRACT

The present invention relates to a process for producing β-carotene in specific solvents.

The present invention relates to a process for producing β-carotene in a specific solvent.

β-Carotene is an organic, strongly coloured red-orange pigment abundant in fungi, plants, and fruits. β-Carotene is an important product with many different ways of application.

β-Carotene is the compound of the following formula (I)

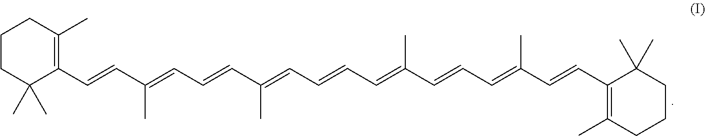 [SMILES: CC1=C(/C=C/C(C)=C/C=C/C(C)=C/C=C/C=C(C)/C=C/C=C(C)/C=C/C2=C(C)CCCC2(C)C)C(C)(C)CCC1]

β-Carotene is a member of the carotenes, which are terpenoids (isoprenoids), synthesized biochemically from eight isoprene units and thus having 40 carbons. Among the carotenes, β-carotene is distinguished by having beta-rings at both ends of the molecule.

β-Carotene is the most common form of carotene found in plants.

When used as a food colouring, it has the E number E160a (ii).

Furthermore, in nature, β-carotene is a precursor (inactive form) to vitamin A via the action of beta-carotene 15,15′-monooxygenase.

β-Carotene is a compound that gives vivid yellow, orange, and red colouring to vegetables. The human body converts β-carotene into vitamin A (retinol).

Next to its dyeing properties β-carotene has also some health benefits, such as effects on eye health, on improved cognitive function, on skin protection and on cancer prevention.

A common way to produce β-carotene is shown in the following scheme

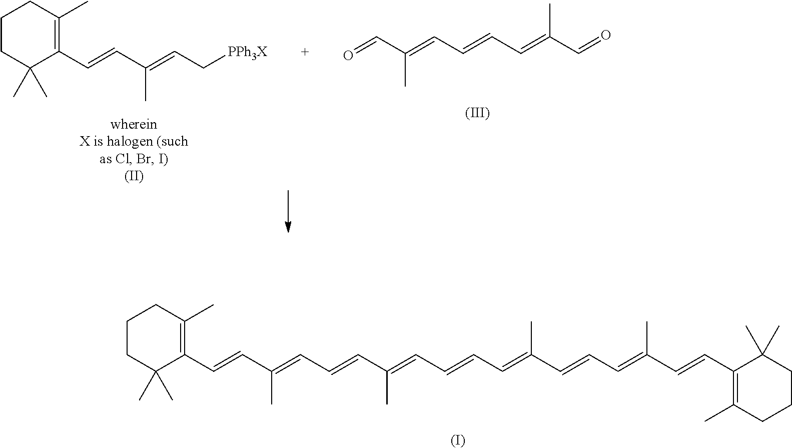

Due to its importance, there is always a need for an improved way to obtain β-carotene.

The phosphonium salt (compound of formula (II)) is charged with the aldehyde component (compound of formula (III)) in the reactor and at least a strong base is added to perform the Wittig reaction.

Quite often it is seen that under these conditions the phosphonium salt is not stable and for this reason a higher amount of phosphonium salt is necessary.

On the other hand, the aldehyde component is not stable towards strong bases which makes it normally impossible to load the aldehyde and the base and dose the phosphonium salt component.

Furthermore, by using alcohols as solvent, which is very common, there is an issue when isolating the product (compound of formula (I), due to fact that a lower alcohols (C₁-C₃-alcohols) are water miscible and therefore difficult to separate.

Surprisingly, we found that the dosage of a solution of the phosphonium salt to a mixture of the aldehyde component and with a specific base and specific solvent result in excellent yield and excellent purity of the resulting β-carotene.

The base (or the mixture of bases) used in the context of the present invention is a compound of formula (IV) and/or a compound of formula (IV′)

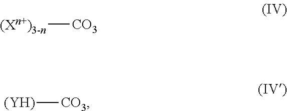

wherein
X is chosen from the group of alkali metals or earth alkali metals, and
n 1 or 2 (it is the charge of the alkali metals or earth alkali metals), and
Y is an alkali metal.

The process according to the present invention is usually carried out in at least one solvent.

The solvent chosen for the process according to the present invention are organic carbonates.

Such organic carbonates have the following formula (V)

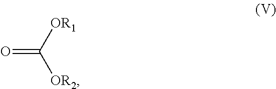

wherein
R₁ is a C₁-C₄alkyl moiety, and
R₂ is a C₁-C₄alkyl moiety.

Therefore, the present invention relates to the process (P) for the production of the compound of formula (I)

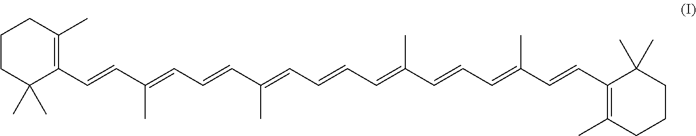 [SMILES: CC1=C(/C=C/C(C)=C/C=C/C(C)=C/C=C/C=C(C)/C=C/C=C(C)/C=C/C2=C(C)CCCC2(C)C)C(C)(C)CCC1]

wherein
a compound of formula (II)

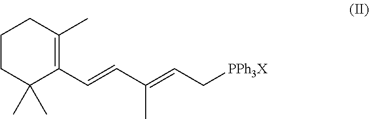

wherein
X is halogen (preferably Cl, Br or I, more preferred Cl)
is reacted with a compound of formula (III)

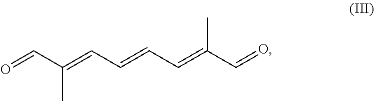

in the presence of at least one compound of formula (IV) and/or a compound of formula (IV′)

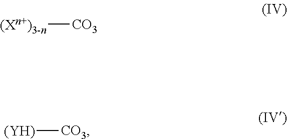

wherein
X is chosen from the group of alkali metals or earth alkali metals, and
n 1 or 2 (it is the charge of the alkali metals or earth alkali metals), and
Y is an alkali metal,
characterised in that at least one compound of formula (V)

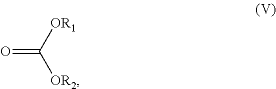

wherein
R₁ is a C₁-C₄alkyl moiety, and
R₂ is a C₁-C₄alkyl moiety,
is used as the solvent.

In the context of the present invention, all disclosed compounds (represented by the chemical formulae) can be in any possible stereochemical configuration.

When using carbonates as solvents, there are no issues with the distillation. Furthermore, carbonates are seen as green solvents.

A preferred process according to the present invention is the one wherein the compound of formula (IV) X is chosen from the group consisting of Ca, Mg, Cs, Li, Na and K.

A preferred process is the one wherein the compound of formula (IV′) Y is chosen from the group consisting of Li, Na and K.

Most preferably, the compounds of formula (IV) and the compounds of formula (IV′) are chosen from the group consisting of CaCO₃, MgCO₃, CS₂CO₃, Li₂CO₃, Na₂CO₃, K₂CO₃, LiHCO₃, NaHCO₃ and KHCO₃.

Therefore, the present invention relates to the process (P1), which is the process (P), wherein at least one the compound of formula (IV), wherein X is chosen from the group consisting of Ca, Mg, Cs, Li, Na and K is used.

Therefore, the present invention relates to the process (P1′), which is the process (P), wherein at least one the compound of formula (IV′), wherein Y is chosen from the group consisting of Li, Na and K is used.

Therefore, the present invention relates to the process (P1″), which is the process (P), wherein the compounds of formula (IV) and the compounds of formula (IV′) are chosen from the group consisting of CaCO₃, MgCO₃, Cs₂CO₃, Li₂CO₃, Na₂CO₃, K₂CO₃, LiHCO₃, NaHCO₃ and KHCO₃.

The process according to the present invention is carried out in the presence of at least one solvent (compound of formula (V)).

Preferably, the process according to the present invention is carried out in the presence of at least one compound of formula (V), wherein
R₁ is a C₁-C₂ alkyl moiety, and
R₂ is a C₁-C₂ alkyl moiety,
as solvent.

More preferably, the process according to the present invention is carried out in the presence of at least one compound of formula (V) chosen from the group consisting of dimethylcarbonate and diethylcarbonate as solvent.

Most preferably, the process according to the present invention is carried out in diethylcarbonate as solvent.

Therefore, the present invention relates to the process (P2), which is the process (P), (P1), (P1′) or (P1″), wherein the process is carried out in the presence of at least one compound of formula (V), wherein
R₁ is a C₁-C₂alkyl moiety, and
R₂ is a C₁-C₂alkyl moiety,
as solvent.

Therefore, the present invention relates to the process (P2′), which is the process (P), (P1), (P1′) or (P1″), wherein the process is carried out in the presence of at least one compound of formula (V) chosen from the group consisting of dimethylcarbonate and diethylcarbonate as solvent.

Therefore, the present invention relates to the process (P2′), which is the process (P), (P1), (P1′) or (P1″), wherein the process is carried out in dimethylcarbonate.

In another embodiment of the present invention, at least one co-solvent (next to the organic carbonate(s)) is used this at least one co-solvent is methanol, ethanol and/or isopropanol.

Preferably the at least one co-solvent is methanol and/or ethanol.

When these co-solvents are used, they are used in an amount of up to 50% (by volume), based on the total volume of the solvent. Usually 2-50% (by volume)

Therefore, the present invention relates to the process (P3), which is the process (P), (P1), (P1′), (P1″), (P2) or (P2′), wherein at least one co-solvent chosen from the group of methanol, ethanol and isopropanol is used.

Therefore, the present invention relates to the process (P3′), which is the process (P), (P1), (P1′), (P1″), (P2) or (P2′), wherein at least one co-solvent chosen from the group of methanol and ethanol is used.

Therefore, the present invention relates to the process (P4), which is the process (P3) or (P3′), wherein at least one co-solvent is used in an amount of up to 50% (by volume), based on the total volume of the solvent.

Therefore, the present invention relates to the process (P4′), which is the process (P3) or (P3′), wherein at least one co-solvent is used in an amount of 2-50% (by volume), based on the total volume of the solvent.

The reaction of the process according to the present invention is usually carried out at a temperature of 0-150° C. Preferably, the process is carried out at 5° C. to 130° C.

Therefore, the present invention relates to the process (P5), which is the process (P), (P1), (P1′), (P1″), (P2), (P2′), (P3), (P3′), (P4) or (P4′), wherein the process is carried out at temperature of 0-150° C.

Therefore, the present invention relates to the process (P5′), which is the process (P5), wherein the process is carried out at a temperature of from at 5° C. to 130° C.

The compound of formula (II) is added to the reaction mixture in an amount of at least 2 mol-eq to the compound formula (III). It can also be added in an excess.

Therefore, the present invention relates to the process (P6), which is the process (P), (P1), (P1′), (P1″), (P2), (P2′), (P3), (P3′), (P4), (P4′), (P5) or (P5′), wherein the compound of formula (II) is added to the reaction mixture in an amount of at least 2 mol-eq to the compound formula (III).

The following examples serve to illustrate the invention. The temperature is given in ° C. and all percentages are related to the weight.

### EXAMPLES

### Example 1

In a 1 liter reactor, C₁₀-dialdehyde (9.03 g), which is the compound of formula (III), and K₂CO₃ anhydrous (37.9 g) were loaded and dimethylcarbonate (324.3 g) was added. The mixture was heated to an internal temperature of 40° C.

After this, vinylsalt (60.1 g, 2.2 eq.), which is the compound of formula (II), was dissolved in MeOH (15.0 g) was added.

After the addition of the vinylsalt solution, the dark red suspension was stirred for 240 min at 40° C. At the end of this time, another 0.02 eq. of vinlysalt (as solution in MeOH) was added.

The reaction mixture was heated to reflux and an azeotropic mixture of dimethylcarbonate and methanol was removed by distillation.

Finally the reaction mixture was cooled to 20° C. and deionized water (150 g) was added. The solids were filtered off.

The solid product was washed with MeOH (90.0 g), water (200 g) and finally again with MeOH (90.0 g).

The dark violet solid was dried under reduced pressure at 40° C. for 8 hours.

Yield: 25.3 g (86.3%) of β-carotene crude as isomeric mixture of all-trans β-carotene (85-92%) and cis-isomers of β-carotene (8-15%).

### Example 2

In a 1 liter reactor, C₁₀-dialdehyde (9.03 g), which is the compound of formula (III), and K₂CO₃ anhydrous (37.9 g) were loaded and a mixture of dimethylcarbonate and methanol (95/5 in wt-%, 324.3 g) was added. The mixture was heated to an internal temperature of 40° C.

After this, vinylsalt (60.1 g, 2.2 eq.), which is the compound of formula (II), dissolved in MeOH (15.0 g) was added.

After the addition of the vinylsalt solution, the dark red suspension was stirred for 240 min at 40° C. At the end of this time, another 0.02 eq. of vinlysalt (as solution in MeOH) was added.

The reaction mixture was heated to reflux and an azeotropic mixture of dimethylcarbonate and methanol was removed by distillation.

Finally the reaction mixture was cooled to IT=20° C. and deionized water (150 g) was added. The solids were filtered off.

The solid product was washed with MeOH (90.0 g), water (200 g) and finally again with MeOH (90.0 g). The dark violet solid was dried under reduced pressure at 40° C. for 8 hours.

Yield: 25.5 g (87.0%) of β-carotene crude as isomeric mixture of all-trans β-carotene (88-92 wt-%) and cis-isomers of β-carotene (8-12 wt-%).

### Example 3

In a 1 liter reactor, C₁₀-dialdehyde (9.03 g), which is the compound of formula (III), and K₂CO₃ anhydrous (37.9 g) were loaded and a mixture of dimethylcarbonate and methanol (324.3 g) was added. The mixture was set to an internal temperature of 10° C.

After this, vinylsalt (60.1 g, 2.2 eq.), which is the compound of formula (II), dissolved in MeOH (35.0 g) was added.

After the addition of the vinylsalt solution, the dark red suspension was stirred for 240 min at 10° C. At the end of this time, another 0.02 eq. of vinlysalt (as solution in MeOH) was added.

The reaction mixture was heated to 40° C. and kept at this temperature for additional 60 min. After this, the suspension was heated to reflux and an azeotropic mixture of dimethylcarbonate and methanol was removed by distillation. Dimethylcarbonate (100-150 ml) was added to the reaction mixture. The reaction mixture is stirred for another 2 h at 80° C.

Finally the reaction mixture was cooled to 20° C. and deionized water (150 g) was added. The solids were filtered off.

Yield: 27.7 g (94.5%) of β-carotene crude as isomeric mixture of all-trans β-carotene (90-94 wt-%) and cis-isomers of β-carotene (6-10 wt-%).

The crude β-carotene (100 g), consisting of an isomeric mixture of 85-94 wt-% of all-trans β-carotene and 6-15 wt-% of β-carotene cis isomers, was suspended in a mixture of MeOH/DMC (330 g, 90/10 as %-w/w). The suspension was heated in an autoclave to 120° C. for 8-16 h.

After this the suspension was cooled to 20° C. and the solid product was filtered off. Yield: 97.0 g (97%) of β-carotene pure as isomeric mixture of all-trans β-carotene (95-98%-w/w) and cis-isomers of β-carotene (2-5%-w/w).

1. Process for the production of to the compound of formula (I) 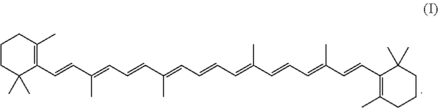 [SMILES: CC1=C(/C=C/C(C)=C/C=C/C(C)=C/C=C/C=C(C)/C=C/C=C(C)/C=C/C2=C(C)CCCC2(C)C)C(C)(C)CCC1]
wherein
a compound of formula (II) 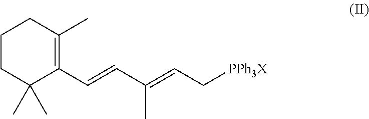
wherein
X is halogen (preferably Cl, Br or I)
is reacted with a compound of formula (III) 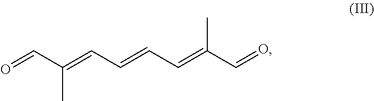
in the presence of at least one compound of formula (IV) and/or a compound of formula (IV′) 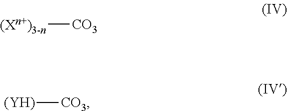
wherein
X is chosen from the group of alkali metals or earth alkali metals, and
n 1 or 2 (it is the charge of the alkali metals or earth alkali metals), and
Y is an alkali metal,
characterised in that at least one compound of formula (V) 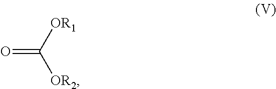
wherein
R₁ is a C₁-C₄alkyl moiety, and
R₂ is a C₁-C₄alkyl moiety,
is used as the solvent.

2. Process according to claim 1, wherein
the compound of formula (IV) X is chosen from the group consisting of Ca, Mg, Cs, Li, Na and K.

3. Process according to claim 1, wherein the compound of formula (IV′) Y is chosen from the group consisting of Li, Na and K.

4. Process according to claim 1, wherein at least one the compound of formula (IV), wherein X is chosen from the group consisting of Ca, Mg, Cs, Li, Na and K is used.

5. Process according to claim 1, wherein at least one the compound of formula (IV′), wherein Y is chosen from the group consisting of Li, Na and K is used.

6. Process according to claim 1, wherein the compounds of formula (IV) and the compounds of formula (IV′) are chosen from the group consisting of CaCO₃, MgCO₃, Cs₂CO₃, Li₂CO₃, Na₂CO₃, K₂CO₃, LiHCO₃, NaHCO₃ and KHCO₃.

7. Process according to claim 1, wherein the process is carried out in at least one the compound of formula (V), wherein
R₁ is a C₁-C₂alkyl moiety, and
R₂ is a C₁-C₂alkyl moiety.

8. Process according to claim 1, wherein the process is carried out in at least one the compound of formula (V) chosen from the group consisting of dimethylcarbonate and diethylcarbonate as solvent.

9. Process according to claim 1, wherein the compound of formula (V) is dimethylcarbonate.

10. Process according to claim 1, wherein at least one co-solvent chosen from the group of methanol, ethanol and isopropanol is used.

11. Process according to claim 10, wherein at least one co-solvent is used in an amount of 2-50% (by volume), based on the total volume of the solvent.

12. Process according to claim 1, wherein the process is carried out at an elevated temperature.

13. Process according to claim 12, wherein the process is carried out at a temperature from 0° C. to 150° C.

14. Process according to claim 1, wherein the compound of formula (II) is added to the reaction mixture in an amount of at least 2 mol-eq to the compound formula (III).
